(19) United States
(12) Patent Application Publication
(10) Pub. No.: US 2026/0241096 A1
(43) Pub. Date: Aug. 20, 2026

(54) INFUSION ELEMENT

(71) Applicant: INREDA DIABETIC B.V., Goor (NL)

(72) Inventors: Robin KOOPS, Goor (NL); Gijs Benno WESTEN, Goor (NL); Mickael Gilbert Robert BOULAY, Goor (NL)

(73) Assignee: INREDA DIABETIC B.V., Goor (NL)

(21) Appl. No.: 19/489,698

(22) PCT Filed: Apr. 19, 2024
(86) PCT No.: PCT/EP2024/060848

§ 371 Date: Dec. 3, 2025

(30) Foreign Application Priority Data
Apr. 20, 2023 (NL) 2034650

(51) Int. Cl. A61M 5/142; A61M 5/172

(52) U.S. Cl. CPC A61M 5/14248; A61M 5/1723; A61M 2230/201

(57) ABSTRACT

An infusion element for infusing a substance into a body of a person, comprising: a housing that is suitable for being attached to the skin of said person; an infusion means for infusing the substance into the body of said person, said infusion means being arranged on and/or protruding from the first surface of the housing; at least one sensor for sensing a characteristic of the person, said sensor being arranged on and/or protruding from the first surface of the housing; wherein in use of the infusion element the infusion means is arranged for infusing said substance at a first depth and said sensor is arranged for sensing the characteristic at a second, different depth. A system for regulating the concentration of glucose in the blood of a person comprising such an infusion element.

The invention relates to an infusion element for infusing a substance into a body of a person, comprising:
a housing that is suitable for being attached to the skin of said person, wherein in use of the infusion element a first surface of the housing is in direct or indirect contact with the skin;
an infusion means for infusing the substance into the body of said person, said infusion means being arranged on and/or protruding from the first surface of the housing;
at least one sensor for sensing a characteristic of the person, said sensor being arranged on and/or protruding from the first surface of the housing;

Such an infusion element is known per se. It is an object of the invention to improve said known infusion element.

This object may be achieved by providing an infusion element according to the preamble, which is characterised in that in use of the infusion element the infusion means is arranged for infusing said substance at a first depth such as to infuse the substance within any of the layers of the skin or below the skin and said sensor is arranged for sensing the characteristic at a second, different depth such as to sense the characteristic in any of the other layers of the skin of the person or below the skin.

The applicant has found that injecting the substance at a different depth than that said characteristic is sensed and in particular that sensing and infusing take place at different layers of or below the skin, has the advantage that interaction between the substance and sensing may be reduced or even prevented, thereby reducing noise in the sensor measurements.

For example, infusing the substance may take place epidermally, i.e. the substance is infused into the epidermis, or dermally, i.e. the substance is infused into the dermis, or subcutaneously, i.e. the substance is infused below the skin.

For example, the substance may be infused at a depth of between 5-10 mm.

For example, sensing the characteristic may take place epidermally, i.e. the characteristic is sensed in the epidermis, dermally, i.e. the characteristic is sensed in the dermis, or subcutaneously, i.e. the characteristic is sensed below the skin.

For example, sensing the characteristic may take place at a depth of about 3 mm.

If the substance is infused epidermally, the characteristic may be sensed dermally or subcutaneously. If the substance is infused dermally, the characteristic may be sensed epidermally or subcutaneously. If the substance is infused subcutaneously, the characteristic may be sensed epidermally or dermally.

For example, the distance between the depth of infusing and the depth of sensing may be between 2-7 mm.

Infusing the substance may take place in any suitable way by means of any suitable infusion means. The infusion means may either be invasive or non-invasive.

For example, in an embodiment of the infusion element according to the invention said infusion means is chosen from the group comprising a cannula, catheter, or needle, wherein said infusion means comprises at least one outlet opening for infusing said substance into said body, wherein said infusion means protrudes from the first surface of the housing, and wherein in use of the infusion element the infusion means is inserted into the body of the person in such a manner that the at least one outlet opening is located at said first depth.

Sensing the characteristic may take place in any suitable way by means of any suitable sensor, for example enzymatic, by means of light, or by means of magnetohydrodynamics. The sensor may either be invasive or not-invasive.

For example, in an embodiment of the infusion element according to the invention the sensor protrudes from the first surface of the housing, wherein in use of the infusion element the sensor is inserted into the body of the person in such a manner that a sensing part of the sensor arranged for sensing said characteristic is located at said second depth.

In an embodiment of the infusion element of the invention the sensor is a printed electronics sensor that is printed on the infusion means or on a film, wherein the infusion means functions as the substrate for the printed electronics sensor or wherein the film functions as the substrate for the printed electronics and wherein the film is provided on the infusion means, respectively.

Such an embodiment provides a practical embodiment in which the infusion means and sensor are combined into one element. Providing the sensor on the infusion means may easily take place by printing or providing the sensor thereon as a printed electronics sensor.

In an embodiment of the infusion element of the invention the sensor is printed or provided on the outer surface of the infusion means.

An advantage hereof is that the curved outer surface of the infusion means provides a relatively large surface for printing or providing the sensor thereon.

Practically at least a sensing part of the printed electronics sensor is printed or provided on the infusion means at a certain location in the length direction of the infusion means, wherein said certain location is at a distance from the outlet opening of the infusion means.

By providing the sensing part of the sensor at a distance from the outlet opening of the infusion means the sensor senses the characteristic at a different depth, i.e. at a different location in a different layer of the skin or below the skin, than that the substance is infused. This way interaction between the substance and sensing may be reduced or even prevented, thereby reducing noise in the sensor measurements.

In an embodiment of the infusion element of the invention the infusion means is made of polyurethane.

Polyurethane is a material that is suitable for infusing said substance and optionally for printing the sensor thereon.

It is noted that the sensor and infusion means may alternatively be combined into one single element in any other different, suitable way.

Alternative to the above described combined sensor and infusion means, the sensor and infusion means may be provided as separate elements that are each arranged on and/or protrude from the first surface of the housing.

Practically the length of the infusion means is longer than the length of the sensor or vice versa.

An advantage of the infusion means and sensor having different lengths is that infusing and sensing may easily take place at different locations within or below the skin.

The sensed characteristic may be any desired characteristic, such as, but not limited, the glucose concentration of the person, for example in the blood or interstitial fluid of the person, the heart rate of the person, the body temperature, the acidity of the blood of the person, ketone bodies in the body of the person, galvanic resistance of the skin of the person. In particular said sensed characteristic may be any characteristic that is indicative for the responsiveness of the person to said substance influencing the blood glucose levels and may therefore require an adaption of the amount of substance influencing blood glucose levels to be provided to that person.

In accordance therewith the at least one sensor is chosen from the group comprising a glucose sensor, a heart rate sensor, a temperature sensor, an acidity or PH sensor, a ketone sensor, a skin resistance sensor.

The invention also relates to a system for regulating the concentration of glucose in the blood of a person, comprising:
a device for selectively supplying at least one and optionally two substances to said person;
at least one and optionally two infusion sets connectable to said device for infusing a said substance to said person, at least one said infusion set comprising an infusion element as described above or below in any one or more of the described embodiments or any suitable combination thereof and/or having any one or more of the above or below described features, alone or in any suitable combination, and comprising a transport element, such as a conduit, tube, hose or the like, that fluidly connects the device to the infusion set, and
a controller for controlling said device such that a certain amount of a said substance is supplied per time unit and thereby infused into the body of the person via said or a respective infusion set.

Such a system in general is known per se and may be used by diabetes patients for regulating the concentration of glucose in their blood. An example of such a system can be found in the international patent application WO2007/049961, but the system may be any other type of a system for regulating the concentration of glucose in the blood of a person. For example, the system may be, but not limited thereto, a closed loop glucose control system, a low glucose suspend system, a hybrid closed loop control system, an insulin pump. In accordance with the invention the system comprises at least one infusion set according to the invention.

The invention will be further explained with respect to figures in which:

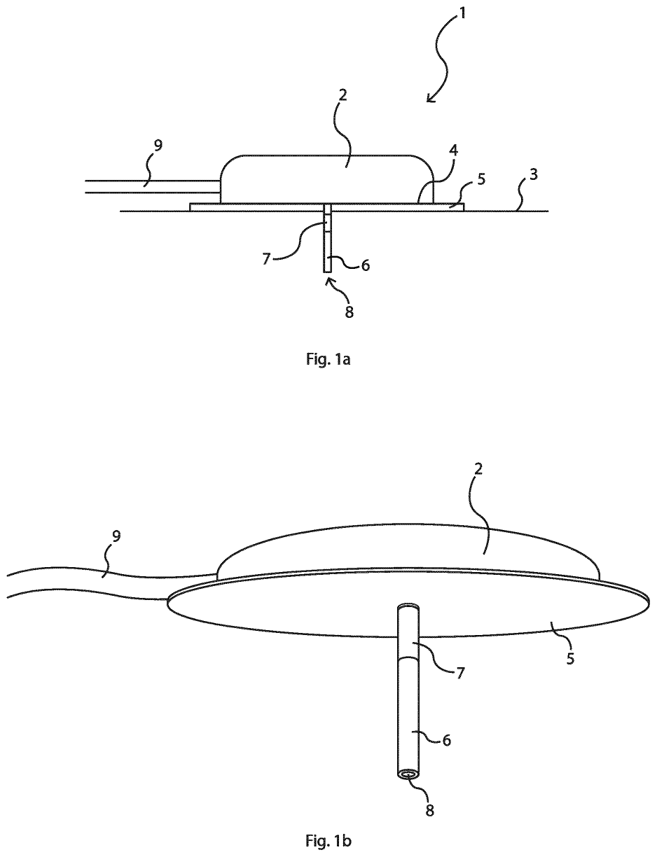

FIGS. 1A and 1B schematically show an infusion element according to a first embodiment of the invention in respectively a side view and a perspective view;

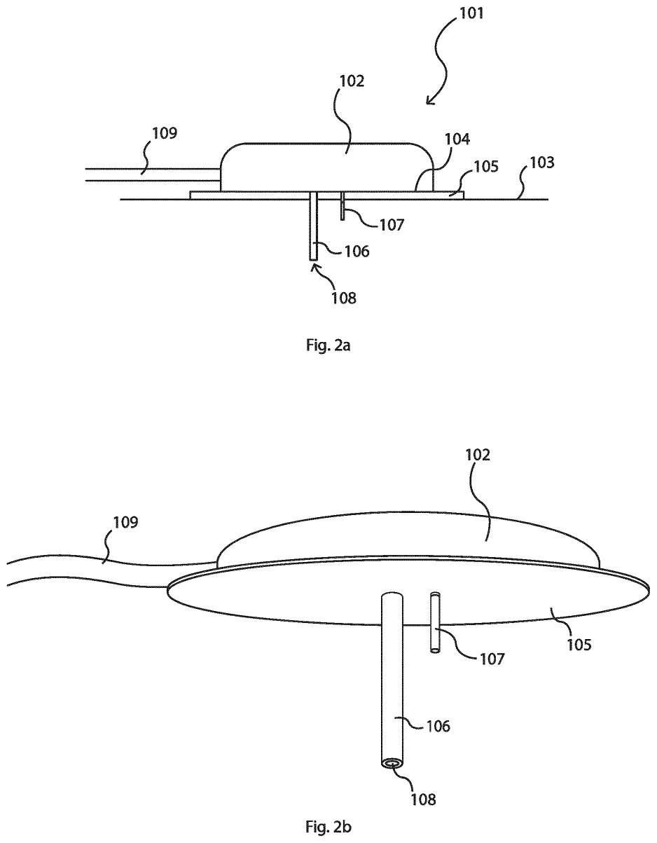

FIGS. 2A and 2B schematically show the infusion element according to a second embodiment of the invention in respectively a side view and a perspective view;

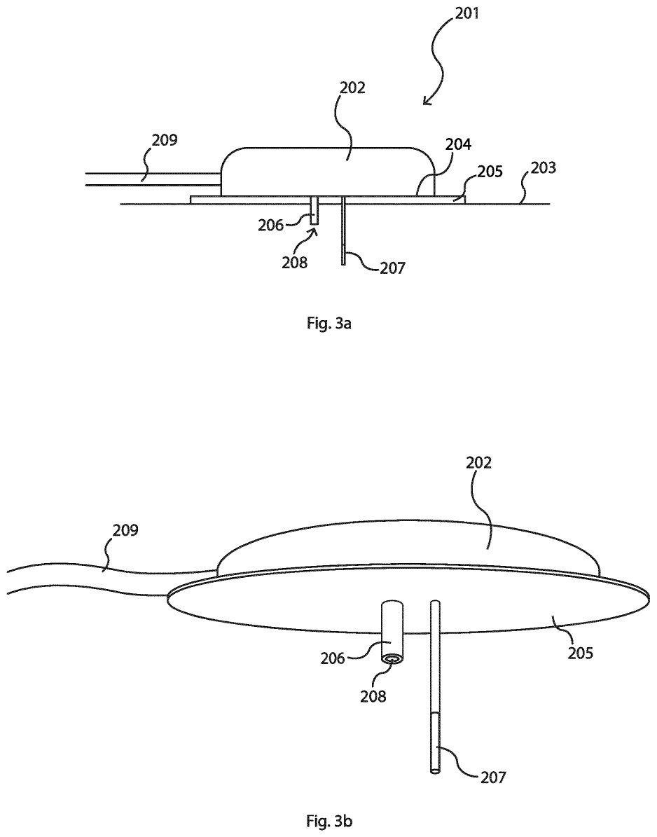

FIGS. 3A and 3B schematically show the infusion element according to a third embodiment of the invention in respectively a side view and a perspective view, and

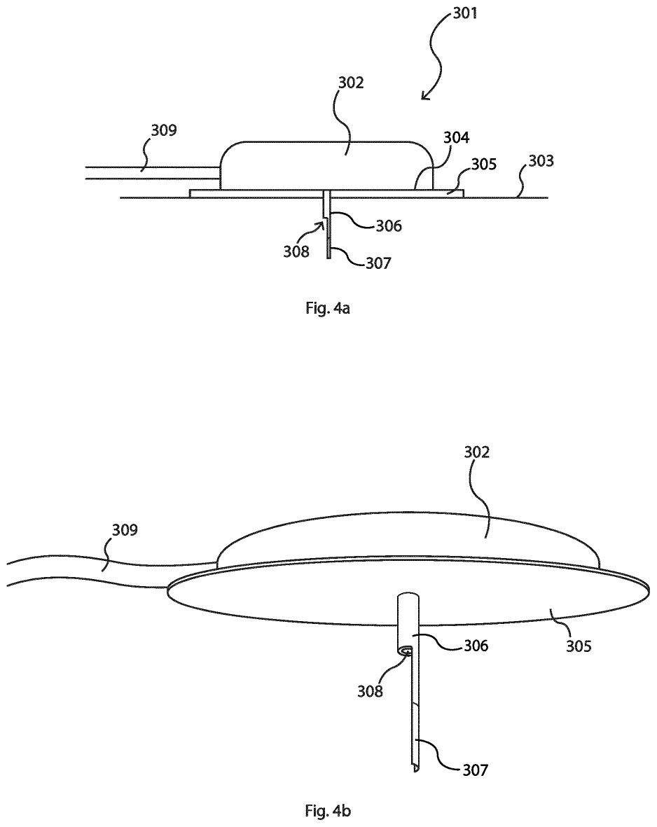

FIGS. 4A and 4B schematically show the infusion element according to a fourth embodiment of the invention in respectively a side view and a perspective view.

In the figures, same or similar features or elements are denoted by same reference numerals increased by one hundred (100). The drawings are schematic and not to scale.

FIGS. 1A and 1B show an infusion element 1 for infusing a substance into a body of a person according to a first embodiment of the invention. In this embodiment, the infusion element 1 comprises a housing 2 that is suitable for being attached to the skin 3 of said person, wherein in use of the infusion element 1 a first surface 4 of the housing 2 is in this case in indirect contact with the skin 3 via an adhesive element 5. The adhesive element 5 attaches the housing 2 to the body by means of its adhesive properties. The infusion element 1 further comprises an infusion means 6 for infusing the substance into the body of said person, wherein in this embodiment the said infusion means 6 protrudes from, i.e. with respect to, the first surface 4 of the housing 2. The infusion element 1 further comprises in this embodiment one sensor 7 for sensing a characteristic of the person. In this embodiment the sensor 7 is a printed electronics sensor that is printed on the outer surface of the infusion means 6, such that the infusion means 6 and sensor 7 form one combined infusion and sensor means. As is visible in the FIGS. 1A and 1B, the sensor 7 or in particular the sensing part thereof is arranged at a distance from an outlet opening 8 of the infusion means 6, such that infusing the substance via the outlet opening of the infusion means takes place at a different depth than sensing the characteristic by means of the sensor 7. In particular, infusing the substance takes place at a first depth, in this embodiment subcutaneously, and sensing the characteristic takes place at a second depth, in this case epidermally or dermally, wherein the first depth is in this embodiment deeper than the second depth.

In the embodiment of FIG. 1 the infusion means 6 is in particular a canula. The canula may be made of polyurethane or any other suitable material for infusing the substance and/or printing the sensor thereon. In this embodiment the canula is the substrate on which the sensor is printed.

Alternatively the sensor may be printed on a film or any other suitable substrate, which film substrate or film is then provided on the canula, for example by attaching the film to the canula, for example by means of an adhesive.

In the embodiment of FIG. 1 the sensor 7 is a glucose sensor for measuring a glucose concentration in the body of the person. The infusion element 1 may comprise communication means for communicating the measured glucose concentration to a device for infusing insulin and/or glucagon as described in further detail below (not shown). For example, the infusion element may comprise a transmitter for transmitting this information to the device or may be connected to the device via a wire. The sensor 7 or at least the sensing part thereof is connected to the transmitter or other communication means (not shown).

The infusion means 6 may for example be used for infusing insulin or glucagon to the person, in order to influence the glucose concentration in the blood of the person. The substance may be supplied to the infusion element 1 via a tube 9, which tube 9 fluidly connects the infusion element 1 to a device for infusing insulin and/or glucagon (not shown). The tube 9 may be releasably connectable to the infusion element 1. Together with the tube 9 the infusion element 1 may form a so-called infusion set. The device may for example be a closed loop glucose control system, a low glucose suspend system, a hybrid closed loop control system, an insulin pump.

FIGS. 2A and 2B show an infusion element 101 for infusing a substance into a body of a person according to a second embodiment of the invention. Only the differences with respect to the first embodiment will be described here. For a further description of the infusion element 101 of FIGS. 2A, 2B the reader is referred to the description of FIGS. 1A, 1B.

The infusion element 101 of FIGS. 2A, 2B differs from the first embodiment of FIGS. 1A, 1B in that the sensor 107 is provided separately from the infusion means 106. As is visible in FIGS. 2A, 2B, both the sensor 107 and the infusion means 106 extend from the first surface 104 and protrude into and optionally below the skin 103 of the person in use of the infusion means 101. The infusion means 106 is longer than the sensor 107, such that infusion takes place at a different depth in or below the skin than sensing. In particular, infusing the substance takes place deeper than sensing. In FIGS. 2A, 2B reference numeral 107 in particular points to the sensing part of the sensor 107.

FIGS. 3A and 3B show an infusion element 201 for infusing a substance into a body of a person according to a third embodiment of the invention. Only the differences with respect to the second embodiment will be described here. For a further description of the infusion element 201 of FIGS. 3A, 3B the reader is referred to the description of FIGS. 2A, 2B, respectively 1A, 1B.

The infusion element 201 of the third embodiment differs from the infusion element 101 of the second embodiment in that the sensor 207 has a longer length than the infusion means 206, such that the substance is infused at less depth than that the glucose concentration is sensed. In particular a sensing part of the sensor 207 is provided near an outer end of the sensor 207, said outer end being the end that is not attached to the housing 202. In FIGS. 3A, 3B reference numeral 207 in particular points to the sensing part of the sensor 207.

FIGS. 4A and 4B show an infusion element 301 for infusing a substance into a body of a person according to a fourth embodiment of the invention. Only the differences with respect to the first embodiment will be described here. For a further description of the infusion element 301 of FIGS. 4A, 4B the reader is referred to the description of FIGS. 1A, 1B.

The infusion element 301 of the fourth embodiment differs from the infusion element 101 of the first embodiment in that the outlet opening 308 is not provided at an outer end of the infusion means 306, but at a distance from the outer end of the infusion means relatively close to the housing 302, the outer end of the infusion means being the end that is not attached to the housing 302. The sensing part of the sensor 307 is arranged near the outer end of the sensor 307. As such, sensing takes place deeper than infusing. In FIGS. 4A, 4B reference numeral 307 in particular points to the sensing part of the sensor 307.

It is noted that the invention is not limited to the shown embodiments but also extends to variants within the scope of the appended claims. For example, a different sensor than a glucose sensor may be provided, alternative or in addition to the glucose sensor that is shown in the figures.

1. An infusion element for infusing a substance into a body of a person, comprising:
a housing that is suitable for being attached to the skin of said person, wherein in use of the infusion element a first surface of the housing is in direct or indirect contact with the skin;
an infusion means for infusing the substance into the body of said person, said infusion means being arranged on and/or protruding from the first surface of the housing; and
at least one sensor for sensing a characteristic of the person, said sensor being arranged on and/or protruding from the first surface of the housing;
wherein in use of the infusion element the infusion means is arranged for infusing said substance at a first depth such as to infuse the substance within any of the layers of the skin or below the skin and said sensor is arranged for sensing the characteristic at a second, different depth such as to sense the characteristic in any of the other layers of the skin of the person or below the skin.

2. The infusion element according to claim 1, wherein said infusion means is chosen from the group comprising a cannula, catheter, or needle, wherein said infusion means comprises at least one outlet opening for infusing said substance into said body, wherein said infusion means protrudes from the first surface of the housing, and wherein in use of the infusion element the infusion means is inserted into the body of the person in such a manner that the at least one outlet opening is located at said first depth.

3. The infusion element according to claim 1, wherein the sensor protrudes from the first surface of the housing, and wherein in use of the infusion element the sensor is inserted into the body of the person in such a manner that a sensing part of the sensor arranged for sensing said characteristic is located at said second depth.

4. The infusion element according to claim 2 wherein the sensor is a printed electronics sensor that is printed on the infusion means or on a film, wherein the infusion means functions as the substrate for the printed electronics sensor or wherein the film functions as the substrate for the printed electronics sensor and wherein the film is provided on the infusion means, respectively.

5. The infusion element according to claim 4, wherein the printed electronics sensor is printed or provided on the outer surface of the infusion means.

6. The infusion element according to claim 4, wherein at least a sensing part of the printed electronics sensor is printed or provided on the infusion means at a certain location in the length direction of the infusion means, wherein said certain location is at a distance from the outlet opening of the infusion means.

7. The infusion element according to claim 1, wherein the infusion means is made of polyurethane.

8. The infusion element according to claim 1, wherein the sensor and infusion means are provided as separate elements that are each arranged on and/or protrude from the first surface of the housing.

9. The infusion element according to claim 8, wherein the length of the infusion means is longer than the length of the sensor or vice versa.

10. The infusion element according to claim 1, wherein the at least one sensor is chosen from the group comprising a glucose sensor, a heart rate sensor, a temperature sensor, an acidity or PH sensor, a ketone sensor, a skin resistance sensor.

11. A system for regulating the concentration of glucose in the blood of a person, comprising:
a device for selectively supplying at least one and optionally two substances to said person;
at least one and optionally two infusion sets connectable to said device for infusing a said substance to said person, at least one said infusion set comprising an infusion element according to claim 1 any and comprising a transport element, such as a conduit, tube, hose or the like, that fluidly connects the device to the infusion element, and
a controller for controlling said device such that a certain amount of a said substance is supplied per time unit and thereby infused into the body of the person via said or a respective infusion set.
